(19) United States
(12) Patent Application Publication
(10) Pub. No.: US 2005/0282763 A1
(43) Pub. Date: Dec. 22, 2005

(54) Methods of treating bladder disorders

(75) Inventor: Mary Lynne Hedley, Lexington, MA (US)

Correspondence Address: FISH & RICHARDSON PC, P.O. BOX 1022, MINNEAPOLIS, MN, 55440-1022, US

(73) Assignee: Zycos Inc., a Massachusetts corporation

(21) Appl. No.: 11/019,955

(22) Filed: Dec. 22, 2004

Related U.S. Application Data
(63) Continuation of application No. 10/074,956, filed on Feb. 12, 2002, now abandoned.
(60) Provisional application No. 60/268,175, filed on Feb. 12, 2001.

(51) Int. Cl. A61K 48/00

(52) U.S. Cl. 514/44; 424/492

(57) ABSTRACT

Methods of treating bladder disorders, including bladder cancer and inflammatory bladder diseases such as interstitial cystitis are disclosed. The methods include identifying a mammal that has or is at risk for having a bladder disorder and administering isolated nucleic acid sequences to the mammal. Nucleic acids used in the methods of the invention contain unmethylated CpG sequences, which are thought to modulate the immune response. Also included are methods that use nucleic acids encoding alpha-MSH. The nucleic acid sequences may be administered individually or together or can be included in the same nucleic acid.

### CROSS REFERENCE TO RELATED APPLICATIONS

This application claims priority from U.S. Provisional Application No. 60/268,175, filed Feb. 12, 2001, the entire content of which is incorporated herein by reference.

### FIELD OF THE INVENTION

The invention relates to methods of treating bladder disorders. Specifically, the methods can be used to treat disorders such as bladder cancer and inflammatory bladder diseases such as interstitial cystitis.

### BACKGROUND OF THE INVENTION

Bladder disorders, including bladder cancer and bladder inflammatory disorders, are on the rise. Bladder cancer is the most common urologic malignancy and was predicted to affect approximately 54,000 people in 1998 (de Vere White & Stapp (1998) Oncology (Huntingt) 12:1717-26). Current treatments include local resection, use of intravesical agents and radical cystectomy (Whittlestone & Persad (2000) Hosp Med 5:336-40). Bladder cancer is the fourth most commonly diagnosed malignancy in men and the eighth most common in women and represents a wide spectrum of disease, ranging from superficial, well-differentiated disease to highly malignant tumors (Metts et al. (2000) J. Matl. Med. Assoc. 92: 285-94). Primary radical surgery remains the standard of care for invasive bladder cancer (Kim & Steinberg (2000) J. Urol. 164(3 Pt 1): 627-32). Transurethral resection (TUR) of the superficial transitional cell carcinoma (TCC) of the bladder is known to be insufficient in controlling the disease because of the unacceptable rates of recurrence, progression and ultimate cystectomy. Adjuvant intravesical chemo-and/or immunotherapy is administered in an effort to enhance the efficacy of surgery alone (Melekos & Moutzouris (2000) Curr Pharm Des 3:345-59).

Intravesical bacillus Calmette-Guerin (BCG) is widely used in the management of bladder cancer, but because it is a living organism, local and disseminated infection may result (Paterson & Patel (1998) Aust N Z J Surg 68: 340-44). The precise mechanism of action of BCG therapy is unknown (Physician's Desk Reference, 1997, Medical Economics Co., Montvale, N.J., p. 1881).

Bladder inflammation may lead to interstitial cystitis (IC). This disorder may initiate with a bacterial infection or antigen assault that damages the bladder lining and renders the lining susceptible to further damage. Inflammation that elicits pain and muscle contraction increases urinary frequency, decreases bladder volume and ultimately affects the ability of urothelial cells to regenerate or the mucosal protective layer to rebuild. Clinical case studies suggest that the impact of IC on quality of life is severe and debilitating. A study of quality of life measures such as physical function, emotional problems, bodily pain, vitality and social function indicated that quality of life among women with the disease is limited, since 4 out of 7 women with IC reported significantly lower quality of life scores (Michael et al. (2000) J. Urol. 164:423-27).

Current treatments of IC include Elmiron® (pentosan polysulphate sodium) and dimethyl sulfoxide (DMSO). Elmiron®, an oral formulation of the glycosaminoglycan (GAG) defensive barrier component of the bladder, may act by rebuilding bladder mucosa. DMSO/hydrocortisone/bicarbonate solutions are instilled into the bladder in an effort to decrease inflammation. DMSO increases penetration of hydrocortisone deep into the bladder wall, where it can be more effective. Early clinical studies suggest that BCG may also be useful in treating IC (Peters et al. (1997) J. Urol. 157:2090-94).

### SUMMARY OF THE INVENTION

The invention is based on the discovery that an isolated nucleic acid may be useful to treat certain bladder disorders in a mammal by modulating the mammal's immune response. In particular, the use of a nucleic acid that contains unmethylated CpG sequences is expected to modulate the immune response of the mammal, e.g., resulting in the switching from a Th2 response to a Th1 response. This modulated immune response can be associated with, for example, reduced antibody production, reduced immune complex deposition in the bladder, decreased histamine release, increased production of certain cytokines, e.g., TNF-α, IL-12 and IL-6. In addition alpha-MSH encoding sequences can be delivered to the mammal, e.g., to decrease inflammation associated with a bladder disorder. More specifically, the invention features a method of modulating an immune response in a mammal, comprising identifying a mammal that has or is at risk for having a bladder disorder and administering an isolated nucleic acid comprising an unmethylated CpG sequence to the mammal, to thereby modulate an immune response in the mammal. In one embodiment, the nucleic acid is delivered to the bladder of the mammal. In a preferred embodiment, the nucleic acid is delivered to the bladder by instillation.

In one embodiment the mammal has bladder cancer. In another embodiment, the mammal has a bladder disorder that is characterized by inflammation. In a preferred embodiment, the inflammation is associated with symptoms of interstitial cystitis. In another preferred embodiment, inflammation is associated with a disruption of the integrity of the bladder lining. In another preferred embodiment, a bacterial infection of the bladder of the mammal is not detected at the time of the administration of the nucleic acid. Examples of bladder inflammation include cystitis induced by antigen challenge in mice and rats (Saban et al. (2000) Am. J. Pathology 156:775-780; Ahluwalia et al. (1998) Br. J. Pharmacol. 124:190-196). Rodent models also aid in the understanding of causes and mechanism of bladder inflammation and can be used to evaluate the usefulness of compounds in humans.

Preferably, the nucleic acid does not encode a naturally occurring polypeptide, such as a viral or bacterial polypeptide. In another embodiment, the nucleic acid is contained within a plasmid. In another embodiment, the nucleic acid is delivered by a microparticle. Preferably, the microparticle comprises a synthetic polymer.

In an embodiment, the nucleic acid further comprises a sequence encoding α-MSH. α-MSH is an anti-inflammatory peptide that can down regulate an immune response. Also within the method, a second isolated nucleic acid can be administered to the mammal, wherein the second isolated nucleic acid encodes α-MSH.

In another embodiment, the invention includes the method of modulating an immune response in a mammal as described herein, wherein administering the isolated nucleic acid results in an amelioration of one or more symptoms of the disorder. In a preferred embodiment, the bladder disorder is bladder cancer and administering the isolated nucleic acid results in a decrease in tumor size or activity. In another preferred embodiment, the bladder disorder is interstitial cystitis and administering the isolated nucleic acid results in a modulation of the immune response from a Th2 to a Th1 response.

In another aspect, the invention includes a method of modulating an immune response in a mammal, comprising identifying a mammal that has or is at risk for having a bladder disorder and administering an isolated nucleic acid comprising a sequence encoding α-MSH to the mammal, to thereby modulate the immune response in the mammal. The nucleic acid sequence encoding α-MSH may be contained within a plasmid. In another embodiment, the nucleic acid encoding α-MSH is contained within or delivered by a microparticle. Preferably, the microparticle comprises a synthetic polymer.

In an embodiment, the method includes administering an isolated nucleic acid comprising a sequence encoding α-MSH to the mammal wherein the bladder disorder is characterized by an inflammation of the bladder. In a preferred embodiment the inflammation is associated with symptoms of interstitial cystitis. In another preferred embodiment, the inflammation is associated with a disruption of the integrity of the bladder lining. In another embodiment, a bacterial infection of the bladder of the mammal is not detected at the time of administration of the nucleic acid. In a further embodiment, the mammal has bladder cancer.

In another aspect, the invention includes an isolated nucleic acid comprising an unmethylated CpG sequence that activates immune cells and a sequence encoding α-MSH. In a further aspect, the invention includes a method of modulating an immune response in a mammal, comprising identifying a mammal that has or is at risk for having a bladder disorder and administering a peptide, e.g., an α-MSH peptide, that binds to a melanocortin receptor to the mammal, to thereby modulate an immune response in the mammal.

“Modulating an immune response” as used herein refers to any change in the immune response in a mammal that is beneficial to the treatment of a bladder disorder. Examples of modulating an immune response include, but are not limited to: redirecting a mammal's immune response from a Th2 to a Th1 response by inducing monocytic and other cells to produce Th1 inducing cytokines; altering the activity of a T cell population to prevent symptoms of a bladder disorder; reducing immunoglobulin (Ig) secretion; reducing immune complex deposition in the bladder; decreasing histamine release in the bladder; and decreasing IL-6 or IL-12 concentration in the urine.

The phrase “identifying a mammal that has or is at risk for having a bladder disorder” refers to any procedure that can be used to determine that a mammal has or is at risk for having a bladder disorder. The phrase encompasses both analysis of the mammal, e.g., by performing diagnostic techniques and/or questioning a patient about symptoms, as well as the receipt of information, e.g., from a patient or from another person, that indicates that the mammal has or is at risk for having a bladder disorder. Examples of methods to detect a bladder disorder in a mammal are well known in the art. For example, urine cytology remains the “gold standard” for identifying bladder cancer (Brown (2000) Urol. Clin. North Am. 27:25-37). Significant prognostic factors for identifying bladder cancer include ploidy status, proliferation index, T stage (clinical tumor stage), lymph node status and histopathologic grade (Lee & Park (1996) Eur. Urol. 29:193-98). Histological grade of cancer can be determined by standards set by the World Health Organization and International Society of Urological Pathology (Cheng et al. (2000) Cancer 88:2326-32) by examining transurethral resection specimens from mammals with tumors. Other methods of identifying mammals at risk for bladder cancer may include the use of biological markers. Potential biological markers include DNA ploidy, S-phase of cells, specific monoclonal antibodies, p53 tumor suppressor gene, retinoblastoma gene (Rb), cell adhesion molecules and angiogenesis (de Vere White & Strapp (1998) Oncology (Huntingt) 12:1717-236). An increase in epidermal growth factor receptor (EGFR) expression may be indicative of bladder cancer (Sierra et al. (2000) Arch. Exp. Urol. 53:323-31). Specific measures obtained during clinical cystoscopy of biopsy materials may also be used as indicators of bladder cancer. These include microvessel density (MVD) and the presence of urinary bladder tumor antigen (BTA TRAK™, Bion Diagnostic Sciences, Redmond, Wash.) (Krapski et al. (2000) BJU Int. 85:1027-32). Specific bladder tumor markers include: bladder tumor antigen (BTA), nuclear matrix proteins, fibrin/fibrinogen degradation products, telomerase (an enzyme that can reconstitute the ends of chromosomes after cell division), hyaluronidase and hyaluronic acid (Brown (2000), supra). Proteomic technologies and use of databases for identifying tumor markers in the urine that may serve as prognostic factors for bladder cancer can also be of use in identification of mammals at risk for bladder disorder. Such tools can be used to reveal and identify proteins that are differentially expressed in tumors and normal urothelium. Thereafter, specific antibodies against the differentially expressed proteins can be used to stain serial cryostat sections of cystectomies from tumor patients (Celis et al. (1999) IUBMB Life 48:19-23; Rasmusen et al. (1996) J. Urol. 155:2113-19).

“Bladder disorders” as used herein is meant to refer to any disorder of the bladder characterized by an infection of the bladder, abnormal cellular growth and/or death of bladder cells, an abnormal bladder-associated immune response, and/or bladder-associated discomfort. The term includes, but is not limited to, bladder inflammation, interstitial cystitis, cystitis, and bladder cancer. Also within the definition is a disorder, e.g., an inflammatory condition, where there is no evidence of ongoing bacterial infection. For example, IC patients typically have no evidence of bacterial infection in their urine at the time of treatment.

“Interstitial cystitis (IC)” refers to a chronic bladder disease of obscure etiology. It commonly affects females, who present with symptoms of pain on bladder filling and urinary frequency. (Peeker et al. (2000) Int Urogynecol J Pelvic Floor Dysfunct 11:290). Bladder inflammation appears to be common in many IC patients. The National Institutes of Health (NIH) have established diagnostic criteria for interstitial cystitis based on the presence of irritative voiding symptoms in the absence of other identifiable pathology (Denson et al. (2000) J Urol 6:1908-11).

Examples of identifying a mammal that has or is at risk for having a bladder disorder where the bladder disorder is IC include: performing cystoscopy; detecting the presence of glomerulations or ulcerations in the bladder; detecting increased numbers of mast cells in the bladder; detecting anti-proliferative factor and increased IL-6 levels in the urine; and administering and evaluating an IC symptom index.

For present purposes, “bladder inflammation” includes disorders associated with, for example, disruption of the integrity of the bladder lining, increased numbers of mast cells in the bladder, histamine release by mast cells in the bladder, presence of anti-proliferative factor in the urine and elevated levels of IL-6 in the urine.

Mammals with bladder inflammation can be generated by sensitization models that induce bladder inflammation, such as antigen sensitization and challenge in the bladder (Saban et al. (2000) Am. J. Pathology 156:775-80; Ahluwalia et al. (1998) Br. J. Pharmacol 124:190-96).

“Treatment” of a bladder disorder as used herein refers to the application of any agent to an individual, wherein the application has the purposes and/or effect of ameliorating the disorder or one or more symptoms associated with the disorder, or diminishing the likelihood of acquiring the disorder in a predisposed individual. In one example, the treatment reduces tumor burden or activity, e.g., in an individual with bladder cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A depicts the structure of the POMC polypeptide, with the various regions indicated below the polypeptide by reference to their amino acid positions.

FIG. 1B depicts the structure of the mini-POMC polypeptide.

FIG. 2 depicts the nucleotide (SEQ ID NO:15) and amino acid (SEQ ID NO:8) sequences of the mini-POMC construct. The α-MSH sequence is underlined in this figure.

FIG. 3 depicts the expression vector pZYC-mini-POMC.

FIG. 4 depicts the structure of α-MSH/serum albumin fusion polypeptides.

### DETAILED DESCRIPTION OF THE INVENTION

The invention encompasses methods for treating bladder disorders. Methods within the invention modulate an immune response in a mammal by identifying a mammal that has or is at risk for having the bladder disorder and administering an isolated nucleic acid to the mammal. Examples of isolated nucleic acids useful in the methods described herein include nucleic acids comprising unmethylated CpG sequences and nucleic acid sequences encoding α-MSH.

Use of Unmethylated CpG Sequences to Treat Bladder Disorders

One aspect of the invention provides for the administration of an isolated nucleic acid containing an unmethylated CpG sequence. “Isolated nucleic acid” refers to a nucleic acid the structure of which is not identical to that of any naturally occurring nucleic acid or to that of any fragment of a naturally occurring genomic nucleic acid spanning more than three separate genes. The term includes, for example, a recombinant DNA that is incorporated into a vector such as an autonomously replicating plasmid or virus. The nucleic acids used in methods described herein can comprise ribonucleotides, deoxyribonucleotides, or modified forms of either nucleotide. Isolated nucleic acid sequences can be single or double stranded and can be polynucleotides or oligonucleotides.

A “CpG sequence” refers to a cytosine followed by a guanine linked by a phosphate bond. The term “unmethylated CpG sequence” refers to a CpG sequence wherein the cytosine is unmethylated. Methylation of the cytosine usually occurs at the 5-position of the pyrimidine ring. CpG sequences that stimulate the immune response and methods of identifying them are described, for example in WO 98/18810, herein incorporated by reference. Examples of nucleic acids containing CpG sequences that may be useful in the invention include TCCATGTCGCTCTGATGCT (SEQ ID NO:1), TCCATGTCGTTCCTGATGCT (SEQ ID NO:2) and TCGTCGTTTTGTCGTTTTGTCGTT (SEQ ID NO:3). CpG sequences are under-represented in vertebrate DNA with respect their expected frequency in a genome with a random sequence.

Plasmid vectors and oligonucleotides containing unmethylated CpG sequences have been shown to modify immune responses more readily than those without these sequences (Sato et al. (1996) Science 273:352-54). Unmethylated CpG sequences can non-specifically activate immune cells to secrete cytokines such as TNFα, IL-12 and IL-6 and thereby re-direct the host immune response from a Th2 to a Th1 phenotype. Inducing Th1 cytokines, such as IFN-γ, may be relevant for treating cancers and certain kinds of inflammatory conditions, such as those associated with immune complexes. Activities of IL-12, IL-6 and TNFα may also be relevant independently of Th1.

Methods of generating nucleic acids containing umnethylated CpG sequences are known in the art and include standard molecular biology techniques. The sequences may be synthesized chemically or biologically. Steps to synthesize small nucleic acids are known in the art. Alternatively, oligonucleotides are commercially available from a wide variety of sources and may be purchased. The oligonucleotides may contain elements to enhance stability such, as phosphorothioate linkages. Bacteria can be transformed with plasmids (autonomous, self-replicating, extrachromosomal, circular DNA molecules) that contain CpG sequences. Auxotrophic or antibiotic resistance genes are used to select bacteria that harbor the plasmid, and large-scale growth and subsequent isolation techniques can then be performed.

CpG containing plasmids can be selected by performing a computer based search for known stimulatory sequences. For example, plasmids with known stimulatory sequences (as identified in WO 98/18810) are compared with a novel plasmid to determine where these stimulatory sequences are located in the novel plasmid. CpG sequences can also be identified experimentally by incubating immune cells (e.g., PBMC) with a nucleic acid and assaying for cytokine production by ELISA, ELISPOT or intracellular staining. Alternately, nucleic acids with known immunostimulatory sequences can be synthesized de novo using any of a number of procedures well known in the art, e.g., the b-cyanoethy phosphoramidite method (Beaucage and Caruthers (1981) Tet. Let. 22:1859) or the nucleoside-H-phosponate method (Garegg et al. (1986) Tet. Let. 27:4051-54; Froehler et al. (1986) Nucl. Acid Res. 14:5399-5407; Garegg et al. (1986) Tet. Let. 27:4055-58; Gaffney et al. (1988) Tet. Let. 29:2619-22). These chemistries can be performed by a variety of commercially available automated oligonucleotide synthesizers. Alternately, CpG dinucleotides can be produced on a large scale in plasmids (see, e.g., Sambrook et al., Molecular Cloning: A Laboratory Manual, Cold Spring Harbor Laboratory Press, N.Y., 1989). Also, nucleic acids may be prepared from existing nucleic acid sequences (e.g., genomic or cDNA) using known techniques, such as those employing restriction enzymes, PCR, exonucleases or endonucleases.

Use of Alpha-MSH Encoding Nucleic Acids to Treat Bladder Disorders

Another aspect of the invention provides for the administration of an isolated nucleic acid encoding a polypeptide comprising α-MSH to a mammal that has or is at risk for having a bladder disorder. As used herein, “α-MSH” refers to a peptide the amino acid sequence of which is SYSMEHFRWGKPV (SEQ ID NO:4) or a fragment thereof that binds to a melanocortin receptor, e.g., MC1-R, with a binding affinity of at least 50% of that possessed by the SYSMEHFRWGKPV (SEQ ID NO:4) peptide. Preferably, the α-MSH peptide includes the amino acid sequence KPV (SEQ ID NO:5) or EHFRW (SEQ ID NO:6).

In one embodiment, the α-MSH peptide induces the generation of regulatory T cells, either in vitro or in vivo. Preferably, the α-MSH peptide possesses at least 50% of an anti-inflammatory activity of the SYSMEHFRWGKPV (SEQ ID NO:4) peptide. Examples of anti-inflammatory activities of the SYSMEHFRWGKPV (SEQ ID NO:4) peptide are reviewed in Lipton and Catania (1997) Immunol. Today 18:140-145, herein incorporated by reference, and include: (1) inhibition of hepatic nitric oxide generation and leukocyte infiltration in mice pretreated with Corynebacterium parvum followed by an injection of lipopolysaccharide; (2) inhibition of the development of chronic inflammation in mycobacterium-induced rats; and (3) decreased histamine release by mast cells.

α-MSH has been shown to be effective in animal models of inflammatory bowel disease, rheumatoid arthritis, and multiple sclerosis (Ceriani et al. (1994) Neuroimmunomodulation 1:28-32; Rajora et al. (1997) Peptides 18:381-85). These conditions are all associated with a Th2 phenotypic response, as characterized by local or systemic cytokine profiles. Isolated nucleic acids that encode α-MSH are designed to secrete α-MSH either for a short term or over an extended period of time. α-MSH administration may have immunomodulatory effects such as: a decrease in production of inflammatory cytokines such as TNF-α; a decrease in mast cell activation and histamine release; a decrease in neutrophil accumulation, e.g., in the bladder; a decreased deposition of immune complexes; decreased production of antibody; and a down-regulation of macrophage activation. If combined with or included as coding sequence within the isolated nucleic acids containing CpG sequences, cytokine profiles of the mammal may also be modulated, e.g., resulting in a change in urinary cytokine profiles from a Th2 (IL-6 high relative to normals) to Th1 (IL-6 low relative to normals) phenotype (Peters et al. (1999) Urology 54:450-53).

Nucleic acids used in methods of the invention can encode a polypeptide that comprises an α-MSH peptide. For example, the polypeptide can comprise an α-MSH peptide (e.g., SYSMEHFRWGKPV (SEQ ID NO:4)) fused to an additional amino acid sequence such as an initiator methioine and/or a trafficking sequence. The nucleic acids may be cloned into an expression vector, i.e., a vector in which the coding sequence is operably linked to expression control sequences. The need for, and identity of, expression control sequences will vary according to the type of cell in which the DNA is to be expressed. Generally, expression control sequences include a transcriptional promoter, enhancer, suitable mRNA ribosomal binding sites, translation start site, and sequences that terminate transcription and translation, including polyadenylation and possibly translational control sequences. Suitable expression control sequences can be selected by one of ordinary skill in the art. The nucleic acids encoding the α-MSH polypeptides described herein may optionally encode a methionine residue at the amino terminus of the polypeptide to facilitate translation. Additionally, an α-MSH expression vector can contain IRES (internal ribosome binding sites) sequences located between nucleic acids encoding polypeptides described herein. This site causes the ribosome to attach to the initiator codon of the downstream translational unit and translate a second protein from a single polycistronic mRNA. For example, a vector can contain IRES sequences between nucleic acid sequences described herein. For a description of IRES sequences and their use, see e.g., U.S. Pat. No. 6,087,129. Standard methods can be used by the skilled person to construct expression vectors (See generally, Sarnbrook et al., 1989, Molecular Cloning: A Laboratory Manual (2nd Edition), Cold Spring Harbor Press, N.Y). Vectors useful in this invention include plasmid vectors, viral vectors, and bacterial vectors. Preferred viral vectors are those derived from retroviruses, adenovirus, adeno-associated virus, pox viruses, SV40 virus, and alpha viruses or herpes viruses.

An example of an expression vector useful in the methods of the invention is a vector that comprises the following nucleic acid sequences: (a) a promoter sequence; (b) a sequence encoding the first 52 amino acids (MPRSCCSRSGALLLALLLQASMEVRGWCLESSQCQDLTTESNLLECIRACKP; SEQ ID NO:7) of the human POMC molecule, comprising a signal sequence encoded by amino acids 1-26 and sorting signal encoded by amino acids 27-52 of POMC; (c) a sequence encoding a joining peptide of POMC or portion of a joining peptide which encodes the natural protease cleavage site (KR) for α-MSH cleavage; (d) a sequence encoding α-MSH; and (e) a stop codon. An example of an amino acid sequence encoded by such a vector is: MPRSCCSRSGALLLALLLQASMEVRGWCLESSQCQDLTTE SNLLECIRACKPREGKRSYSMEHFRWGKPV (SEQ ID NO:8).

A nucleic acid can encode an α-MSH concatamer. An α-MSH concatamer is a polypeptide containing two or more α-MSH peptides. The α-MSH units of the concatamer may optionally include additional amino acid sequences between the units and/or flanking the units. This arrangement of α-MSH peptides can include any number of α-MSH units, e.g., at least 2, 3, 4, 5, 10, 20, 30, 40, 50, 100, 200, or more α-MSH units.

The α-MSH units of an α-MSH concatamer can be separated by a linker sequence. The linker sequence need not be of any defined length. A linker sequence separating α-MSH units can include an amino acid sequence that functions as a protease cleavage site (an amino acid sequence that can be specifically recognized and cleaved by a protease). For example, an α-MSH concatamer containing five α-MSH units, each unit linked to another by a linker sequence comprising a protease cleavage site, can be cleaved by a protease to release five individual α-MSH units, with or without leftover linker residues at one end or the other. Additionally, some of the linkers may not comprise a protease cleavage site, resulting in one or more intact α-MSH concatamers following protease cleavage. The linker sequence between two α-MSH units can optionally contain two or more protease cleavage sites. For example, the linker can include a protease cleavage site that cleaves immediately adjacent to the carboxy end of a first α-MSH unit, followed by a stretch of amino acids, followed by another protease cleavage that cleaves immediately adjacent to the amino end of a second α-MSH unit. This results in the release of α-MSH units having few if any linker amino acids remaining attached to the α-MSH sequence.

The protease cleavage site can be a site recognized by a cell-associated protease or a serum protease. Cell associated proteases include membrane proteins, membrane-associated proteins, and cytosolic proteins. An example of a protease cleavage site recognized by a cell-associated protease is the amino acid sequence recognized by furin, a cell-associated protease found in the trans golgi.

As an alternative (or in addition) to facilitating protease-induced cleavage, the linker sequence can provide spacing and/or orientation to the respective α-MSH units to promote the biological functioning of the individual α-MSH units in the context of an intact α-MSH concatamer. The linker sequence should separate the α-MSH units by a distance sufficient to ensure that each α-MSH unit properly folds into its secondary structure. Preferred linker sequences (1) should adopt a flexible extended conformation, (2) should not exhibit a propensity for developing an ordered secondary structure that could interact with the functional α-MSH units, and (3) should have minimal hydrophobic or charged character, which could promote undesired interaction with the functional α-MSH units. Typical surface amino acids in flexible protein regions include Gly, Asn and/or Ser. Permutations of amino acid sequences containing Gly, Asn and Ser would be expected to satisfy the above criteria for a linker sequence. Other near-neutral amino acids, such as Thr and Ala, can also be used in the linker sequence.

A linker sequence length of 20 amino acids can be used to provide a suitable separation of functional protein domains, although longer or shorter linker sequences may also be used. The length of the linker sequence separating the α-MSH units is generally greater than 3, and preferably greater than 4 amino acids: for example, from 5 to 500 amino acids, or more preferably from 5 to 100 amino acids Preferably, the linker sequence is about 5-30 amino acids. In preferred embodiments, the linker sequence is about 5 to about 20 amino acids, and is advantageously about 10 to about 20 amino acids. Amino acid sequences useful as linkers of the α-MSH units include, but are not limited to, (SerGly₄; SEQ ID NO:16)_{y} wherein y is at least 2, or Gly₄SerGly₅Ser (SEQ ID NO:17). A preferred linker sequence has the formula (SerGly4)₄ (SEQ ID NO:18). Another preferred linker has the sequence ((Ser₄Gly)₃SerPro) (SEQ ID NO:19).

Alternatively, the α-MSH units can be directly fused without a linker.

A polypeptide including an α-MSH concatamer can further include a trafficking sequence. A “trafficking sequence” is an amino acid sequence that causes a polypeptide to which it is fused to be transported to a specific compartment of the cell. The term “trafficking sequence” is used interchangeably with “targeting sequence” herein. The trafficking sequence can be included in the polypeptide in the presence or absence of a linker sequence between α-MSH units and/or between the trafficking sequence and an α-MSH unit. The trafficking sequence is used to direct the α-MSH concatamer to a specific compartment of the cell and/or to be secreted by the cell. In one example, a signal sequence or leader is fused to the α-MSH concatamer, thereby directing the fusion polypeptide to the endoplasmic reticulum (ER) during translation. The signal sequence is cleaved from the polypeptide in the ER, resulting in the mature form of the protein, e.g., a secreted protein. An example of a useful signal sequence that can be fused to the α-MSH concatamer is the signal sequence of the pro-opiomelanocortin (POMC) polypeptide (MPRSCCSRSGALLLALLLQASMEVRG; SEQ ID NO:9) or a portion thereof that directs the secretion of the polypeptide when expressed in a mammalian cell. Preferably, the portion of the POMC signal sequence includes a fragment of the POMC signal sequence at least five amino acids in length. Other useful signal sequences include the signal peptide of HLA-DRα (MAISGVPVLGFFIIAVLMSAQESWA; SEQ ID NO:10) and the signal peptide of the Adenovirus E3 protein.

Other examples of trafficking sequences that can be fused to the α-MSH concatamer include an amino acid sequence that guides a polypeptide to an endosome (e.g., the trafficking sequence of the invariant chain), to a secretory granule (e.g., the POMC sorting sequence (WCLESSQCQDLTTESNLLECIRACKP; SEQ ID NO:11)), and to a lysosome (e.g., KFERQ (SEQ ID NO:12), QREFK (SEQ ID NO:13), and other pentapeptides having Q flanked on one side by four residues selected from K, R, D, E, F, I, V, and L).

A polypeptide containing an α-MSH concatamer can further include a membrane sequence. The membrane sequence, typically a region largely made up of hydrophobic and other uncharged amino acid residues, permits the insertion of the polypeptide of which it is a component into a membrane. The membrane sequence can optionally span the membrane. The polypeptide can further include a signal sequence, e.g., a signal sequence described herein. The fusion polypeptide can optionally contain a membrane sequence that comprises stretch of hydrophobic amino acids at its amino terminus that function as an uncleaved signal for translocation into the endoplasmic reticulum and anchoring of the protein in the membrane, e.g., the hydrophobic transmembrane domains of type II transmembrane proteins such as the invariant chain (Ii), Ly-49, CD23, CD69, hepatic lectins, influenza virus neuraminidase, and intestinal isomaltase.

When inserted into the plasma membrane, the α-MSH concatamer can be oriented either extracellularly or intracellularly. Alternatively, the polypeptide can include one or more α-MSH units on either side of the membrane sequence, so that the resultant polypeptide can have extracellular and intracellular α-MSH units.

The polypeptide can include a linker sequence between the membrane sequence and an α-MSH unit, a linker sequence between α-MSH units, or both. A linker sequence can include an amino acid sequence that functions as a protease cleavage site, as described herein. Alternatively, the linker sequence can be designed to orient the α-MSH units in a manner that promotes their biological activity.

The membrane sequence of the fusion polypeptide can be any sequence that can be anchored in a membrane, thereby maintaining the membrane attachment of the polypeptide of which the membrane sequence is a component. A membrane sequence includes a sequence of at least about 15 amino acid residues, e.g., about 20, 25, 30, 35, 40, or 45 residues, which are inserted in the membrane. Membrane sequences are rich in hydrophobic residues, and typically have an alpha-helical structure. In a preferred embodiment, at least 50%, e.g., at least 60%, 70%, 80%, 90%, 95% or even all of the amino acids of a membrane domain are hydrophobic, e.g., leucines, isoleucines, tyrosines, or tryptophans. Membrane domains are described in, for example, Zagotta et al. (1996) Annual Rev. Neurosci. 19: 235-263. The membrane sequence can correspond to all or a portion of the membrane domain of a naturally occurring protein, e.g., a human protein, e.g., the membrane domain of the transferrin receptor. A “portion of a membrane domain” means a sequence of consecutive amino acids contained in the membrane domain of a naturally occurring protein, wherein the portion retains the ability to maintain the polypeptide of which it is a component anchored in or associated with the cell membrane. Alternatively, the membrane sequence can be a variant of a naturally occurring membrane domain (i.e., with one or more substituted residues) or portion thereof, or can be a completely artificial amino acid sequence.

Polypeptides including an α-MSH concatamer linked to a membrane sequence can further include a cytoplasmic domain. A αcytoplasmic domain” refers to an amino acid sequence that is fused to a membrane sequence and that, when the polypeptide is anchored in the membrane of a cell, ends up in the cytoplasmic side of the cell. The cytoplasmic domain can be of any length and can be derived from a naturally occurring protein or can be an artificial sequence. A cytoplasmic domain can control the regulation or sorting of the polypeptide. For example, the cytoplasmic domain of the invariant chain (Ii) contains sequences that cause sorting of Ii to an endosome. Other cytoplasmic domains can participate in cell signaling processes.

A polypeptide containing an α-MSH concatamer can further include a glycosylphosphatidylinositol (GPI) attachment signal peptide. A “GPI attachment signal peptide” is an amino acid sequence that directs replacement of itself by a Preassembled GPI in the ER. The GPI attachment signal peptide at the carboxy terminus of a GPI-linked protein is replaced by a preassembled GPI in the ER by a transamidation reaction, through the formation of a carbonyl intermediate. Many eukaryotic cell surface proteins are anchored to the cell membrane via a GPI linkage. The GPI attachment signal peptides of Thy-1 (see GenBank™ Accession Number P04216) and CD24 (see GenBank™ Accession Number A48996) are examples of GPI attachment signal peptides that may optionally be linked to the α-MSH concatamer described herein.

Anchoring a polypeptide to the membrane via linkage to GPI permits cleavage of the polypeptide from the membrane by phosphatidylinositol-specific phospholipases. For example, when a cell containing, attached to its plasma membrane, an α-MSH concatamer fused to a GPI moiety is treated with phosphatidylinositol-specific phospholipase C (PI-PLC), cleavage and release of the α-MSH concatamer will occur. The polypeptide can also contain a linker sequence between α-MSH units of the concatamer and/or between the GPI moiety and the α-MSH concatamer. A linker sequence can include an amino acid sequence that functions as a protease cleavage site, as described herein. For example, a polypeptide can include five α-MSH units linked to each other by a linker comprising a protease cleavage site, wherein the carboxy terminal α-MSH unit is fused to a GPI attachment signal peptide.

In another aspect of the invention, a polypeptide can include α-MSH fused to a membrane sequence. This polypeptide includes a single α-MSH, rather than the multiple units contained in the α-MSH concatamer described above. This polypeptide can, for example, have a length of less than 45 amino acids (e.g., less than 40, 35, 30, 25, or 20). This α-MSH/membrane sequence fusion can have any of the properties described above for the fusion of an α-MSH concatamer and a membrane sequence. For example, the α-MSH/membrane sequence fusion polypeptide can further include a signal sequence or other trafficking sequence. Additionally, the fusion polypeptide can include a linker sequence, e.g., located between the membrane sequence and α-MSH. This linker sequence can include an amino acid sequence that can function as a protease cleavage site.

A polypeptide can also include α-MSH fused to a trafficking sequence. This trafficking sequence can have any of the properties described above for the fusion of an α-MSH concatamer and a trafficking sequence. In one example, the trafficking sequence is not the POMC signal sequence. If the trafficking sequence is the POMC signal sequence, then the fusion polypeptide preferably does not include the entire POMC sequence, and most preferably consists of (a) the POMC signal sequence, (b) the POMC sorting sequence, (c) α-MSH, and (d) optionally a sequence that serves as a linker between (b) and (c). An example of such a fusion polypeptide is MPRSCCSRSGALLLALLLQASMEVRG WCLESSQCQDLTTESNLLECIRACKPREGKRSYSMEHFRWGKPV (SEQ ID NO:8).

The trafficking sequence, e.g., a signal sequence, can correspond to the sequence of a secreted peptide, e.g., serum albumin, e.g., human or murine serum albumin, or a portion thereof that directs secretion of the polypeptide. The fusion polypeptide can have a length of less than 100 amino acids, such as less than 90, 80, 70, 60, 50, 40, 30, or 20 amino acids. The fusion polypeptide can include a linker sequence, e.g., located between the trafficking sequence and α-MSH. In one example, a fusion polypeptide contains an albumin sequence or a portion thereof that promotes secretion of the polypeptide, a linker sequence, e.g., GGVGG (SEQ ID NO:14), and α-MSH. A linker sequence can optionally include an amino acid sequence that can function as a protease cleavage site. In one example, the fusion polypeptide contains (a) mouse serum albumin signal peptide, (b) mouse serum albumin signal propeptide, (c) mouse serum albumin, (d) α-MSH, and optionally (e) sequences that serve as linker and protease cleavage sites between (c) and (d). Additionally, human serum albumin signal peptide and human serum albumin signal propeptide can be used.

In a polypeptide that includes a trafficking sequence, e.g., a signal sequence, and α-MSH, the α-MSH need not be directly linked to the trafficking sequence. As described above, the trafficking sequence and the α-MSH can be separated by a linker. Additionally, the α-MSH can be inserted, e.g., by recombinant DNA technology, within all or a portion of the amino acid sequence of a secreted protein, e.g., a secreted protein having a signal sequence. Preferably, the α-MSH is positioned in the fusion polypeptide in what corresponds to an exposed portion of the secreted protein, such as a solvent accessible loop of the secreted protein, e.g., the α-MSH is not inserted in a portion of the amino acid sequence of the secreted protein that is buried such as an α helix or β pleated sheet. The fusion polypeptide can optionally include protease cleavage sites that flank the α-MSH peptide to promote cleavage of α-MSH from the fusion polypeptide.

Many fusion polypeptides can be made between a serum albumin or a fragment thereof and an α-MSH peptide. For example, α-MSH can be fused to the carboxy terminus of murine or human serum albumin. A linker can be included between the serum albumin sequence and the α-MSH sequence. The linker can include a protease recognition site such as a furin cleavage site. In another example, α-MSH can be inserted in a solvent accessible loop of albumin, e.g., human or murine serum albumin. The three-dimensional structure of serum albumin demonstrates that the protein contains at least four solvent accessible loops. α-MSH sequences could be placed in one, two, three, or four of these loops. The resulting fusion polypeptide can be used as a source of biologically active α-MSH. The fusion polypeptide can optionally include protease cleavage sites that flank the α-MSH sequences. In this way, a single fusion protein can be used to enhance the number of α-MSH molecules that can be produced.

A nucleic acid can encode α-MSH fused to a GPI attachment signal peptide. The GPI attachment signal peptide can have any of the properties described above for the fusion of an α-MSH concatamer and a GPI attachment signal peptide. The polypeptide can include a linker sequence, e.g., a sequence comprising a protease cleavage site, between α-MSH and the GPI attachment signal peptide. Alternatively, the polypeptide can include a linker without a protease cleavage site or no linker at all between α-MSH and the GPI attachment signal peptide. In this embodiment, α-MSH can be cleaved from the cell membrane by the action of, e.g., PI-PLC.

Also within the invention is an isolated nucleic acid comprising an unmethylated CpG sequence that stimulates and/or activates immune cells and an α-MSH encoding sequence, e.g., an α-MSH encoding sequence described herein. Activation of immune cells includes, for example, the induction of proliferation and/or production of cytokines by NKβ cells; immunoglobulin proliferation and/or production by B cells; and secretion of cytokines by antigen presenting cells (e.g., dendritic cells, macrophages, and monocytes).

An isolated nucleic acid used in a method of the invention may contain an unmethylated CpG sequence and a sequence encoding α-MSH as described herein. Alternatively, an isolated nucleic acid containing an unmethylated CpG sequence can be delivered separately or in conjunction with a second isolated nucleic acid encoding α-MSH as described herein.

Delivery of Nucleic Acids

The invention encompasses systems and methods for the delivery of nucleic acids to a mammal that has or is at risk for having bladder disorder. These delivery systems allow for targeting of the compositions to the sites where their activity is most beneficial. For example, the nucleic acids used in methods of the invention can be delivered intravesicularly into the bladder of the animal.

In addition to local administration to the bladder, nucleic acids described herein can be administered to subjects in any manner known in the art, e.g., intramuscularly, intravenously, intraarterially, intradermally, intraperitoneally, intranasally, intravaginally, intrarectally or subcutaneously, or they can be introduced into the gastrointestinal tract, the mucosa, or the respiratory tract, e.g., by inhalation of a solution or powder containing microspheres. The nucleic acids described herein can be administered using standard methods, e.g., those described in Donnelly et al. (1994) J. Inun. Methods 176:145 and Vitiello et al. (1995) J. Clin. Invest. 95:341. Additionally, the compositions described herein may be administered via a gene gun or via electorporation. Administration can be local (e.g., delivery to the bladder) or systemic.

The nucleic acids can be naked or associated or complexed with a delivery vehicle. For a description of the use of naked DNA, see e.g., U.S. Pat. No. 5,693,622, herein incorporated by reference. The nucleic acids can be delivered in conjunction with pharmaceutically acceptable carriers. A “pharmaceutically acceptable carrier” is intended to include a substance that can be coadministered with a nucleic acid or a nucleic acid delivery complex and that allows the nucleic acid to perform its indicated function. Examples of such carriers include solutions, solvents, dispersion media, delay agents, emulsions, saline, lipid, depot systems, liposomes, particulates, virus-like particles, microspheres, or nanospheres; as colloidal suspensions; or as powders. Nucleic acids and polypeptides can be delivered using delivery vehicles known in the art, such as lipids, liposomes, ISCOMS, microspheres, microcapsules, microparticles, gold particles, virus-like particles, nanoparticles, polymers, hydrogels, condensing agents, polysaccharides, polyamino acids, dendrimers, saponins, adsorption enhancing materials, or fatty acids. Viral particles can also be used, e.g., retroviruses, adenovirus, adeno-associated virus, pox viruses, SV40 virus, alpha virus or herpes viruses.

It is expected that a dosage of approximately 1 to 200 μg of DNA would be administered per kg of body weight per dose. As is well known in the medical arts, dosage for any given animal depends upon many factors, including the animal's size, body surface area, age, the particular compound to be administered, sex, time and route of administration, general health, and other drugs being administered concurrently. Determination of optimal dosage is well within the abilities of a scientist or pharmacologist of ordinary skill in the art.

Microparticles, e.g., those described in U.S. Pat. No. 5,783,567, can be used as vehicles for delivering macromolecules such as DNA, RNA, or polypeptides into cells. Microparticles may also be made, for example, according to the methods of Mathiowitz, et al. as described in WO 95/24929, herein incorporated by reference. The microparticles can contain macromolecules embedded in a polymeric matrix or enclosed in a shell of polymer. Alternately, the macromolecules can be delivered by being complexed or attached to a polymeric matrix or a shell of a polymer as well as being embedded in the polymeric matrix or enclosed in a shell of polymer. Microparticles act to maintain the integrity of the macromolecule, e.g., by maintaining the DNA in a nondegraded state. Microparticles can also be used for pulsed delivery of the macromolecule, and for delivery at a specific site or to a specific cell or target cell population. Most preferably, microparticles containing nucleic acids described herein are delivered intravesicularly into the bladders of animals.

The polymeric matrix can be a synthetic or natural biodegradable co-polymer such as poly-lactic-co-glycolic acid, starch, gelatin, or chitin. Microparticles that are less than 10 μM in diameter can be used in particular to maximize delivery of DNA molecules into a subject's phagocytotic cells. Alternatively, microparticles that are greater than 10 μM in diameter can be delivered to the bladder, where they form a deposit. As the deposit breaks down, the nucleic acid and associated pieces of the deposit are released gradually over time and taken up by neighboring cells.

The compositions described herein can be used to effect a wide variety of immunomodulatory functions, e.g., anti-inflammatory functions. The compositions can be used to inhibit the activity of various cells and/or molecules of the immune system. Compositions can be delivered to the bladder, for example, by catheterizing a mammal that has or is at risk for having bladder disorder. For example, tubing is passed through the urethra to the bladder, allowing the instillation of nucleic acids directly to the bladder. Various types of bladder disorders can be treated according to these methods, including bladder cancer and IC. The nucleic acids can be delivered to modulate an immune response, as described herein. For example, the α-MSH compositions can be used to inhibit the following: histamine release from mast cells; neutrophil chemotaxis and/or migration to an inflamed site; macrophage activation, and; expression of costimulatory factors, e.g., CD86 and/or CD40, by dendritic cells.

The following examples are meant to illustrate, but not limit, the invention.

### EXAMPLES

### Example 1

### Construction of MiniPOMC Expression Vectors

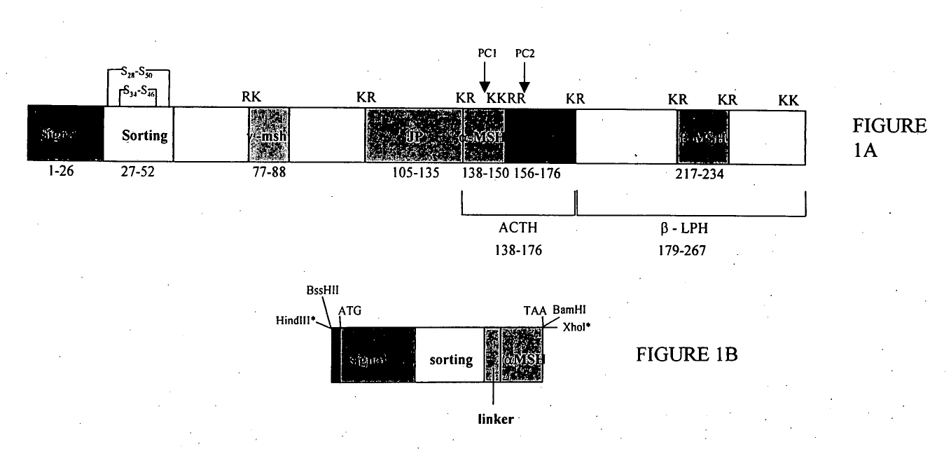

The structure of the POMC polypeptide is depicted in FIG. 1A. The location of various regions and features of POMC are indicated by reference to specific amino acid residues indicated below the depiction of the polypeptide. A polypeptide consisting of POMC amino acid sequences 1-26, 27-52, 138-150, and a linker sequence is depicted in FIG. 1B. This polypeptide has been designated miniPOMC. The construction of nucleic acids encoding miniPOMC is described in this example.

Oligonucleotides encoding the human POMC signal peptide, the human POMC sorting peptide, a partial junction peptide, and the α-MSH sequence SYSMEHFRWGKPV (SEQ ID NO:4) were synthesized in vitro. The POMC oligonucleotides were constructed based upon the human POMC cDNA sequence (GenBank™Accession NM_{—}000939).

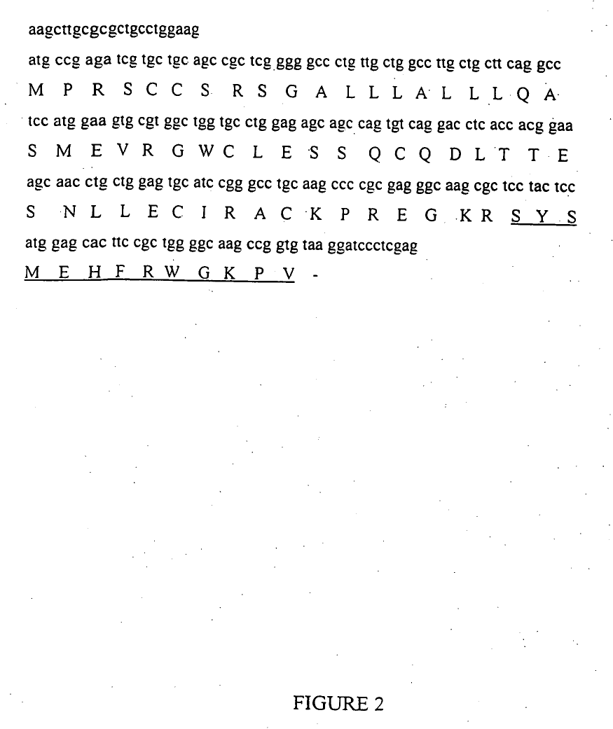

The synthesized oligonucleotides were annealed and subcloned into HindIII and Xhol sites of pCMV-Script (Stratagene, San Diego) to generate the plasmid pCMV-miniPOMC. Both the nucleotide (SEQ ID NO:15) and amino acid (SEQ ID NO:8) sequences of the miniPOMC construct are depicted in FIG. 2. The following restriction enzyme sites were incorporated into the cDNA construct via oligonucleotide synthesis: HindIII and BssHII sites were placed upstream of the start codon; and BamHI and Xhol sites were placed downstream of the stop codon.

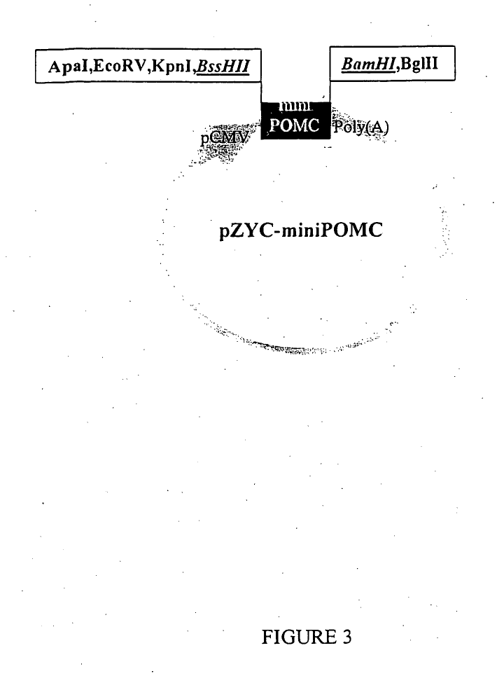

The miniPOMC construct (FIG. 2) was excised from pCMV-miniPOMC by a BssHII and BamHI digest, and subcloned into the BssHII and BamHI sites of the expression vector pZYC to generate pZYC-miniPOMC (FIG. 3). pZYC is a modification of the pBIOTOPE plasmid described in U.S. Pat. No. 6,013,258.

Two copies of the miniPOMC construct (FIG. 2) were subcloned into the vector pIRES (Clontech, Calif.) at multiple cloning sites A and B to generate the vector pIRES-2X miniPOMC. The pIRES-2X miniPOMC expression vector was generated as follows. pIRES was digested with NheI and the ends were blunted using the large (Klenow) fragment of DNA Polymerase I (BioLabs, Mass.). The resulting vector was further digested with XhoI. A miniPOMC fragment was excised from pCMV-miniPOMC by EcoRV (blunt) and Xhol digests. This fragment was then cloned into the blunt-XhoI pIRES vector to generate pIRES-miniPOMC. pIRES-miniPOMC was then digested with NotI, treated with Klenow to create a blunt end, and then further digested with XmaI. A miniPOMC fragment was removed from pCMV-miniPOMC, digested with XhoI, blunted with Klenow, and then further digested with XmaI. The XhoI-XmaI miniPOMC fragment was then cloned into the blunt-XmaI pIRES-miniPOMC vector to generate pIRES-2X miniPOMC.

### Example 2

### Construction of α-MSH/Serum Albumin Fusion Polypeptides

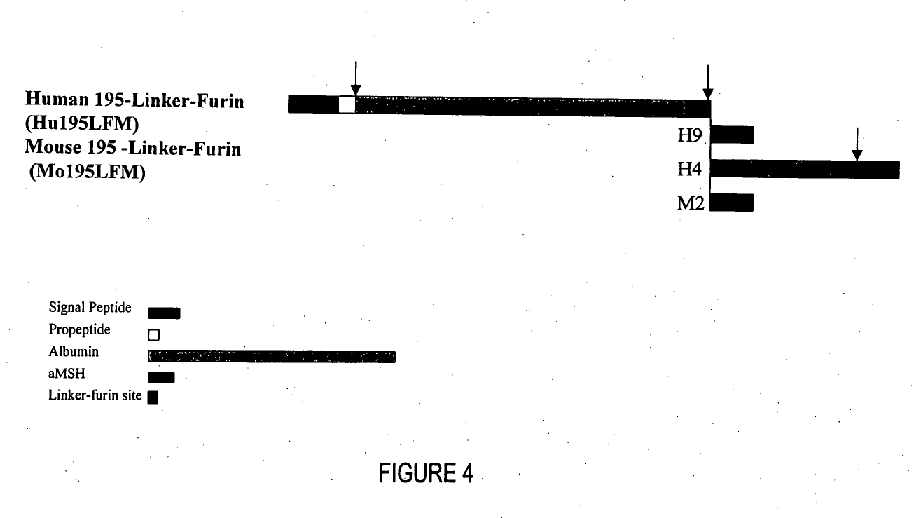

Three α-MSH/serum albumin fusion polypeptides are depicted in FIG. 4. The constructs contain: (a) mouse serum signal peptide (MKWVTFLLLLFVSGSAFS; SEQ ID NO:20) or human serum albumin signal peptide (MKWVTFISLLFLFSSAYS; SEQ ID NO:21); (b) mouse or human serum albumin propeptide (RGVFRR; SEQ ID NO:22); (c) the first 195 amino acids of the mouse serum albumin

| | |
|---|---|
| (EAHKSEIAHRYNDLGEQHFKGLVLIAFSQYLQKCSY | (SEQ ID NO:4) |
| DEHAKLVQEVTDFAKTCVADESAANCDKSLHTLFGDK | |
| LCAIPNLRENYGELADCCTKQEPERNECFLQHKDDNP | |
| SLPPFERPEAEAMCTSFKENPTTFMGHYLHEVARRHP | |
| YFYAPELLYYAEQYNEILTQCCAEADKESCLTPKLDG | |
| VKEKALVSSRV; SEQ ID NO:23) | |
| or | |
| human serum albumin | |
| (DAHKSEVAHRFKDLGEENFKALVLIAFAQYLQQCPF | |
| EDHVKLVNEVTEFAKTCVADESAENCDKSLHTLFGDK | |
| LCTVATLRETYGEMADCCAKQEPERNECFLQHKDDNP | |
| NLPRLVRPEVDVMCTAFHDNEETFLKKYLYEIARRHP | |
| YFYAPELLFFAKRYKAAFTECCQAADKAACLLPKLDE | |
| LRDEGKASSAK; SEQ ID NO:24); | |
| (d) a linker | |
| (GGYGG; SEQ ID NO:25); | |
| (e) a furin site | |
| (RIRR; SEQ ID NO:26); | |
| and | |
| (f) an α-MSH sequence | |
| SYSMEHFRWGKPV. | |

The linker and furin site sequences are introduced by PCR-based site-directed mutagenesis into the previously constructed serum albumin-α-MSH constructs. Briefly, two homologous long primers are designed; the sequences for the linker and the furin site are at the center, flanked by at least 25 bases homologous to the human or mouse serum albumin and α-MSH. During the PCR reaction, the primers anneal to the circular plasmid (at the homologous flanking sequences), while the non-homologous (linker and furin) sequences loop out. The polymerase extends the sequences, and this non-logarithmic PCR gives rise to circular product. The PCR reaction is then digested with DpnI, a restriction enzyme that recognizes a four base pair consensus sequence on a methylated template. Therefore, only the PCR product is left intact, and it is subsequently transformed into competent bacteria (DH5α cells). Colonies are picked for DNA preparation. Because a new restriction site is engineered in the primer, clones can be chosen after digest with that restriction enzyme (e.g., BamHI). The DNA is then sequenced for final confirmation.

The amino acid sequences of the three constructs depicted in FIG. 4 are as follows.

| | |
|---|---|
| H9: MKWVTFISLLFLFSSAYSRGVFRRDAHKSEVA | (SEQ ID NO:27) |
| HRFKDLGEENFKALVLIAFAQYLQQCPFEDHVKLVN | |
| EVTEFAKTCVADSESAENCDKSLHTLFGDKLCTVAT | |
| LRETYGEMADCCAKQEPERNECFLQHKDDNPNLPRL | |
| VRPEVDVMCTAFHDNEETFLKKLYEIARRHPYFYAP | |
| ELLFFAKRYKAAFTECCQAADKAACLLPKLDELRDE | |
| GKASSAKGGYGGRIRRSYSMEHFRWGKPV | |
| H4: MKWVTFISLLFLFSSAYSRGVFRRDAHKSEVA | (SEQ ID NO:28) |
| HRFKDLGEENFKALVLIAFAQYLQQCPFEDHVKLVN | |
| EVTEFAKTTCVADESAENCDKSLHTLFGDKLCTVAT | |
| LRETYGEMADCCAKQEPERNECFLQHKDDNPNLPRL | |
| VRPEVDVMCTAFHDNEETFLKKYLYEIARRHPYFYA | |
| PELLFFAKRYKAAFTECCQAADKAACLLPKLDELRD | |
| EGKASSAKGGYGGRIRRSYSMEHFRWDEFKASSAKG | |
| GYGGRIRRSYSMEHFRWGKPV | |
| M2: MKWVTFLLLLFVSGSAGSRGVFRREAHKSEIA | (SEQ ID NO:29) |
| HRYNDLGEQHFKGLVLIAFSQYLQKCSYDEHAKLVQ | |
| EVTDFAKTCVADESAANCDKSLHTLFGDKLCAIPNL | |
| RENYGELADCCTKQEPERNECFLQHKDDNPSLPPFE | |
| RPEAEAMCTSFKENPTTFMGHYLHEVARRHPYFYAP | |
| ELLYYAEQYNEILTQCCAEADKESCLTPKLDGVKEK | |
| ALVSSVRGGYGGRIRRSYSMEHFRWGKPV | |

### Example 3

### Instillation into the Bladder and Measurement of the Inflammatory Response in Mice

Cystitis is induced in mice as follows. Groups of 16 mice are sensitized intraperitoneally with 1 μg of dinitrophenyl (DNP₄) human serum albumin in 1 mg of alum on days 0, 7, 14 and 21. The protocol induces sustained levels of immunoglobulin E (IgE) up to 56 days post-sensitization. One week after the last sensitization, cystitis is induced by antigen challenge intravesicularly with 150 μl DNP₄-ovalbumin (1 μg/ml). Control mice are given 150 ul of saline. To ensure consistent contact of substances with the bladder, infusion is repeated twice within a 30 minute interval. Cystitis is measured between 1 and 10 days post-sensitization by examination of histological sections for infiltration of inflammatory cells and degranulated mast cells (Saban et al. (2000) Am. J. Pathology 156:775-80). Mast cell number and activation can be measured using morphological analysis (Saban, et al. supra). Histological evaluation demonstrates that bladders isolated from sensitized mice challenged with saline do not present any sign of inflammation or edema. Sensitized mice challenged with antigen (antigen-induced cystitis) develop an inflammatory response characterized by vasodilation, edema, intense polymorphonuclear neutrophil (PMN) infiltration in the mucosa and submucosal layers and activation of resident mast cells in the bladder. This inflammatory response is characterized by acute inflammation based on the strong vascular component, predominance of PMNs, and near absence of macrophages/monocytes.

Prior to or following induction of cystitis, microspheres containing isolated nucleic acids or nucleic acids devoid of microspheres are introduced into the bladder via catheterization. Sensitized mice are anesthetized (ketamine, 40 mg/kg, and xylazine, 2.5 mg/kg i.p)., then transurethrally catheterized (24 Ga, ¾ in; Angiocath, Becton Dickinson, Sandy, Utah), and the urine is drained by applying slight digital pressure to the lower abdomen. A metal guide wire is used to pass intravenous tubing through the urethra and into the bladder. A thin pipette tip is then used at the end of the tubing to instill microspheres into the urethra (Haller et al. (2000) Biomedical Engineering Society Annual Conference, Seattle, Wash.); The urinary bladders are instilled with either 150 μl of saline, saline containing 5-500 μg nucleic acid, or microspheres with 5-500 μg of nucleic acids. The bladders are instilled at a slow rate to avoid trauma and vesicoretaral reflux. Between 1 and 10 days after the instillation of nucleic acids, mice are sacrificed with pentobarbital (20 mg/kg i.p.), and bladders are removed rapidly and fixed in buffered formalin for histology (n=8/group). Histological sections are examined for infiltration of inflammatory cells and degranulated mast cells.

### Example 4

### Instillation into the Bladder and Measurement of Inflammatory Response in Rats

Female Wistar rats (150-175 g) are sensitized by intraperitoneal injection of a mixture of 1 mg ovalbumin (OA) and 100 mg aluminum hydroxide suspended in 1 ml of saline. Fourteen days later, rats are anesthetized with pentobarbitone (45 mg kg⁻¹, i.p.) for intravesical administration of OA to induce interstitial cystitis by antigen challenge. The urinary bladder is manually emptied of urine and then catheterized via the urethra by means of a polyethylene tube (PE 50, Clay Adams) inserted to 2 cm from the external urethral orifice. The animals receive 0.3 ml of OA (10 mg OA in sterile saline) or the vehicle control (saline) over 10 seconds and the catheter is allowed to remain in place for a further 10 seconds before withdrawal. The severity of inflammation is measured by examination of plasma protein extravasation (PPE) at various time points using the Evans blue technique (Saria & Lundberg (1983) Eur. J. Pharmacol. 235:211-19). Briefly, Evans blue (50 mg/kg, i.v.) is administered and 5 minutes later the dye is flushed out of the cardiovascular system by perfusion with warm (37° C.) saline (25 ml/min for 2 minutes) via intracardiac puncture: the right atria are incised to allow the expulsion of the perfusion medium. The end point of each experiment is considered the starting of the intracardiac perfusion procedure. The urinary bladder is then excised; after removal of extraneous tissue, the bladder is washed in saline and blotted dry before weighing. The Evans blue content is determined by extraction of the Evans blue from each bladder in 100% formamide (4 ml) at 60° C. for 24 hours. The dye content is determined by the measurement of absorbance at 620 nm with an automated microplate reader EL 312 (Bio-Tek Instruments, U.S.A). The amount of dye extravasated in each bladder is determined from an Evans blue standard curve and expressed as ng/mg of tissue weight (Ahluwalia et al. (1998) Br. J. Pharmacol 124:190-96).

Prior to or following induction of disease, rats are anesthetized with pentobarbital (45 mg kg⁻¹, i.p.) for intravesical administration of microspheres containing nucleic acids described herein. The animals receive 0.3 ml of sterile saline containing microspheres with 5-500 μg of nucleic acids or 5-500 μg nucleic acids in saline or the vehicle control (saline) over 10 seconds, and the catheter is allowed to remain in place for a further 10 seconds before withdrawal.

Plasma protein extravasation (PPE) by the Evans blue technique is measured in sensitized animals treated with nucleic acids or saline control at various times (1-10 days) after microsphere treatment.

### Example 5

### Measurement of Contractile Activity of the Bladder in Rats

Contractile activity of the bladder in rats after induction of IC is measured following administration of nucleic acids described herein. Following treatment, rats are sacrificed and their bladders are removed and cleared of extraneous tissue. Longitudinal strips of the bladder are mounted in organ baths and contractile activity is recorded with Basile isotonic transducers. Preparations are first equilibrated by measuring constant responses to Neurokinin A, 0.1 μm (30 minute washing intervals between responses). After equilibration, the bladder is subjected to OA (1 mg/ml) and the response is measured (Ahluwalia et al. supra).

### Example 6

### Instillation into the Bladder and Measurment of Inflammatory Response in Humans

Subjects with interstitial cystitis are given 50 ml of a placebo, microspheres containing nucleic acids described herein, or nucleic acids described herein in a pharmaceutically acceptable carrier instilled into the bladder via a Foley catheter. The clamped catheter is left in place and the subjects are asked to retain the solution for 2 hours, changing body position every 15 minutes. If the subject becomes too uncomfortable during the instillation, the bladder is temporarily drained, the patient is allowed to recover and the bladder contents are reinstilled at a volume comfortable for the subject. Alternately, patients can be catheterized and microparticles containing isolated nucleic acids or nucleic acids in a pharmaceutically acceptable carrier can be sprayed onto the bladder lining with a specialized device (e.g., a spray gun).

After 6 weekly instillations are complete, the subjects return for followup at 1, 2, 3 and 6 months, and for a final evaluation when all subjects have been followed for at least 6 months. The effectiveness of the treatment with nucleic acids described herein is assessed by one or more of the following tests, in examples 6-8.

### Example 7

### Measurement of Anti-Proliferative Factor and Heparin Binding Epidermal Growth Factor in the Urine of Humans Treated with Nucleic Acids

Measurement of antiproliferative factor in humans with IC can be performed, for example, by the methods of Chai et al. (2000) J. Urol. 163:1440-44. Urine from IC patients contains an anti-proliferative factor that inhibits urothelial proliferation and contains decreased levels of he parin-binding epidermal growth factor-like growth factor (HB-EGF) compared to controls. Urine is collected immediately before and 2 to 4 hours and two weeks after treatment with nucleic acids described herein. Urinary HB-EGF is measured by enzyme-linked immunosorbent assay and urinary antiproliferative factor activity is determined by measuring ³H-thymidine uptake by normal bladder urothelial cells.

### Example 8

### Measurement of Cytokines in the Urine of Mammals Treated with Nucleic Acids

Commercially available or prepared ELISAs are used to determine levels of urinary cytokines IL-2, IL-4, IL-6, IL-8, IL-10, IL-12, tumor necrosis factor, human granulocyte-macrophage colony stimulating factor, IL-1 beta and/or interferon-gamma in the urine of humans or other mammals treated with nucleic acids described herein relative to controls. Results indicative of a decreased inflammatory response include decreases in IL-6 and IL-8 relative to controls.

### Example 9

### Measurement of Subiect Improvement Following Treatment with Nucleic Acids

Qualified questionnaires can be used to determine the efficacy of treatment of IC patients. Survival indexes and reduction in metastases can be used in assessing the treatment with cancer patients. A Symptom Index and Problem Index for IC developed by O'Leary et al. ((1997) Urology 49(Supplement 54):58-63) measures urinary and pain symptoms and assesses how problematic symptoms are for patients with IC.

### Example 10

### Use of Isolated Nucleic Acids to Treat Bladder Cancer

Isolated nucleic acids described herein may be used to treat bladder cancer, alone or in combination with other treatments, e.g. surgery. Patients are treated with isolated nucleic acids described herein using methods described for IC patients (e.g., catheterization or spraying the bladder). Cytokine levels in blood and urine are measured prior to and 24 hours following treatment. Certain cytokine levels (e.g., IL-6) are expected to decrease in response to treatment.

Although the invention has been described with reference to the presently preferred embodiments, it should be understood that various modifications can be made without departing from the spirit of the invention. Accordingly, the invention is limited only by the following claims.

1. A method of modulating an immune response in a mammal, comprising:
identifying a mammal that has or is at risk for having a bladder disorder; and
administering an isolated nucleic acid comprising an unmethylated CpG sequence to the mammal, to thereby modulate an immune response in the mammal.

2. The method of claim 1, wherein the nucleic acid is delivered to the bladder of the mammal.

3. The method of claim 2, wherein the nucleic acid is delivered to the bladder by instillation.

4. The method of claim 1, wherein the nucleic acid does not encode a naturally occurring polypeptide.

5. The method of claim 1, wherein the nucleic acid is contained within a plasmid.

6. The method of claim 1, wherein the nucleic acid is delivered by microparticles.

7. The method of claim 6, wherein the microparticle comprises a synthetic polymer.

8. The method of claim 8, wherein the microparticle comprises a synthetic polymer.

9. The method of claim 1, wherein the mammal has a bladder disorder that is characterized by inflammation.

10. The method of claim 9, wherein the inflammation is associated with symptoms of interstitial cystitis.

11. The method of claim 9, wherein the inflammation is associated with a disruption of the integrity of the bladder lining.

12. The method of claim 1, wherein a bacterial infection of the bladder of the mammal is not detected at the time of the administration of the nucleic acid.

13. The method of claim 1, wherein the mammal has bladder cancer.

14. The method of claim 1, wherein the nucleic acid further comprises a sequence encoding α-MSH.

15. The method of claim 1, further comprising administering a second isolated nucleic acid to the mammal, wherein the second isolated nucleic acid encodes α-MSH.

16. The method of claim 1, wherein the mammal has a bladder disorder, and wherein administering the isolated nucleic acid results in an amelioration of one or more symptoms of the disorder.

17. The method of claim 16, wherein the bladder disorder is bladder cancer and wherein administering the isolated nucleic acid results in a decrease in tumor size or activity.

18. The method of claim 16, wherein the bladder disorder is interstitial cystitis and wherein administering the isolated nucleic acid results in a modulation of the immune response from a Th2 response to a Th₁ response.

19. A method of modulating an immune response in a mammal, comprising:
identifying a mammal that has or is at risk for having a bladder disorder; and
administering an isolated nucleic acid comprising a sequence encoding α-MSH to the mammal, to thereby modulate an immune response in the mammal.

20. The method of claim 19, wherein the nucleic acid is contained within a plasmid.

21. The method of claim 19, wherein the nucleic acid is contained within a microparticle.

22. The method of claim 21, wherein the microparticle comprises a synthetic polymer.

23. The method of claim 19, wherein the nucleic acid is delivered by a microparticle.

24. The method of claim 23, wherein the microparticle comprises a synthetic polymer.

25. The method of claim 19, wherein the mammal has a bladder disorder that is characterized by an inflammation of the bladder.

26. The method of claim 25, wherein the inflammation is associated with symptoms of interstitial cystitis.

27. The method of claim 25, wherein the inflammation is associated with a disruption of the integrity of the bladder lining.

28. The method of claim 19, wherein a bacterial infection of the bladder of the mammal is not detected at the time of the administration of the nucleic acid.

29. The method of claim 19, wherein the mammal has bladder cancer.

30. An isolated nucleic acid comprising an unmethylated CpG sequence and a sequence encoding α-MSH, wherein the unmethylated CpG sequence comprises an immunostimulatory sequence.

31. A method of modulating an immune response in a mammal, comprising:
identifying a mammal that has or is at risk for having a bladder disorder; and
administering a peptide that binds to a melanocortin receptor to the mammal, to thereby modulate an immune response in the mammal.

32. The method of claim 31, wherein the peptide is an α-MSH peptide.


SEQUENCE LISTING (29 sequences)

SEQ ID NO: 1 - DNA, 19 bp, Homo sapiens
tccatgtcgctctgatgct

SEQ ID NO: 2 - DNA, 20 bp, Homo sapiens
tccatgtcgttcctgatgct

SEQ ID NO: 3 - DNA, 24 bp, Homo sapiens
tcgtcgttttgtcgttttgtcgtt

SEQ ID NO: 4 - protein, 13 aa, Homo sapiens
SYSMEHFRWGKPV

SEQ ID NO: 5 - protein, 3 aa, Homo sapiens
KPV

SEQ ID NO: 6 - protein, 5 aa, Homo sapiens
EHFRW

SEQ ID NO: 7 - protein, 52 aa, Homo sapiens
MPRSCCSRSGALLLALLLQASMEVRGWCLESSQCQDLTTESNLLECIRACKP

SEQ ID NO: 8 - protein, 70 aa, Artificial Sequence
  Synthetically generated peptide
MPRSCCSRSGALLLALLLQASMEVRGWCLESSQCQDLTTESNLLECIRACKPREGKRSYSMEHFRWGKPV

SEQ ID NO: 9 - protein, 26 aa, Homo sapiens
MPRSCCSRSGALLLALLLQASMEVRG

SEQ ID NO: 10 - protein, 25 aa, Homo sapiens
MAISGVPVLGFFIIAVLMSAQESWA

SEQ ID NO: 11 - protein, 26 aa, Homo sapiens
WCLESSQCQDLTTESNLLECIRACKP

SEQ ID NO: 12 - protein, 5 aa, Homo sapiens
KFERQ

SEQ ID NO: 13 - protein, 5 aa, Homo sapiens
QREFK

SEQ ID NO: 14 - protein, 5 aa, Artificial Sequence
  Linker sequence
GGVGG

SEQ ID NO: 15 - DNA, 247 bp, Artificial Sequence
  CDS (23)...(232): Synthetic construct
aagcttgcgcgctgcctggaagatgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagccccgcgagggcaagcgctcctactccatggagcacttccgctggggcaagccggtgtaaggatccctcgag

SEQ ID NO: 16 - protein, 10 aa, Homo sapiens
SGGGGSGGGG

SEQ ID NO: 17 - protein, 11 aa, Homo sapiens
GGGGSGGGGGS

SEQ ID NO: 18 - protein, 20 aa, Homo sapiens
SGGGGSGGGGSGGGGSGGGG

SEQ ID NO: 19 - protein, 17 aa, Homo sapiens
SSSSGSSSSGSSSSGSP

SEQ ID NO: 20 - protein, 18 aa, Mus musculus
MKWVTFLLLLFVSGSAFS

SEQ ID NO: 21 - protein, 18 aa, Homo sapiens
MKWVTFISLLFLFSSAYS

SEQ ID NO: 22 - protein, 6 aa, Homo sapiens
RGVFRR

SEQ ID NO: 23 - protein, 195 aa, Mus musculus
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVR

SEQ ID NO: 24 - protein, 195 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAK

SEQ ID NO: 25 - protein, 5 aa, Artificial Sequence
  Linker sequence
GGYGG

SEQ ID NO: 26 - protein, 4 aa, Artificial Sequence
  Furin site
RIRR

SEQ ID NO: 27 - protein, 241 aa, Homo sapiens
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKGGYGGRIRRSYSMEHFRWGKPV

SEQ ID NO: 28 - protein, 268 aa, Homo sapiens
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKGGYGGRIRRSYSMEHFRWDEGKASSAKGGYGGRIRRSYSMEHFRWGKPV

SEQ ID NO: 29 - protein, 241 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRGGYGGRIRRSYSMEHFRWGKPV